(19) United States
(12) Patent Application Publication
(10) Pub. No.: US 2025/0258151 A1
(43) Pub. Date: Aug. 14, 2025

(54) GAS SENSOR

(71) Applicant: TDK Corporation, Tokyo (JP)

(72) Inventor: Kei TANABE, Tokyo (JP)

(21) Appl. No.: 19/196,872

(22) Filed: May 2, 2025

Related U.S. Application Data
(63) Continuation of application No. PCT/JP2022/042803, filed on Nov. 18, 2022.

(51) Int. Cl. G01N 33/00; G01N 27/18; H05B 1/02

(52) U.S. Cl. CPC G01N 33/004; G01N 27/18; H05B 1/0297; H05B 2203/022

(57) ABSTRACT

Disclosed herein is a gas sensor that includes: first and second thermistors; first and second heaters configured to heat the first and second thermistors, respectively; and a control circuit. The control circuit is configured to, in a first period, generate an output signal based on a detection signal appearing at a connection point between the first and second thermistors by making the heating temperature of the second heater higher than the heating temperature of the first heater. The control circuit is configured to, in a second period, make the heating temperature of the first heater higher than the heating temperature of the second heater and switch the connection relation so as to make an amount of a current flowing through the first thermistor and an amount of a current flowing through the second thermistor different from each other.

### BACKGROUND OF THE ART

### Field of the Art

The present disclosure relates to a gas sensor.

### Description of Related Art

International Publication WO 2020/031517 discloses a gas sensor configured to calculate the concentration of a gas to be detected based on the level of a detection signal appearing at the connection point of two series-connected thermistors. The gas sensor described in International Publication WO 2020/031517 heats a thermistor constituting a detection element and a thermistor constituting a reference element to 150° C. and 300° C., respectively, to acquire a detection signal and then heats the thermistor as the detection element and thermistor as the reference element to 300° C. and 150° C., respectively, to eliminate a difference in thermal history between the two thermistors.

However, even in the gas sensor described in International Publication WO 2020/031517, a slight difference in thermal history occurs between the two thermistors.

### SUMMARY

A gas sensor according to the present disclosure includes: a detection circuit including a first thermistor and a second thermistor; a first heater configured to heat the first thermistor; a second heater configured to heat the second thermistor; and a control circuit configured to control a connection relation in the detection circuit and heating temperatures of the first and second heaters. The control circuit is configured to, in a first period, generate an output signal indicating a concentration of a gas to be measured based on a detection signal appearing at a connection point between the first thermistor and the second thermistor by connecting the first and second thermistors in series and heating the first and second heaters such that the heating temperature of the second heater is higher than the heating temperature of the first heater. The control circuit is configured to, in a second period, heat the first and second heaters such that the heating temperature of the first heater is higher than the heating temperature of the second heater and switch the connection relation so as to make an amount of a current flowing through the first thermistor and an amount of a current flowing through the second thermistor different from each other.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 1 according to a first embodiment of the technology described herein;

FIG. 2 is a timing chart for explaining the operation of the gas sensor 1;

FIG. 3 is a graph illustrating the temperature characteristics of the thermistors Rd1 and Rd2;

FIG. 4 is a circuit diagram illustrating the configuration of a gas sensor 2 according to a second embodiment of the technology described herein; and

FIG. 5 is a timing chart for explaining the operation of the gas sensor 2.

### DETAILED DESCRIPTION OF THE EMBODIMENTS

The present disclosure describes a gas sensor that recues a difference in thermal history between two thermistors.

Some embodiments of the present disclosure will be explained below in detail with reference to the accompanying drawings.

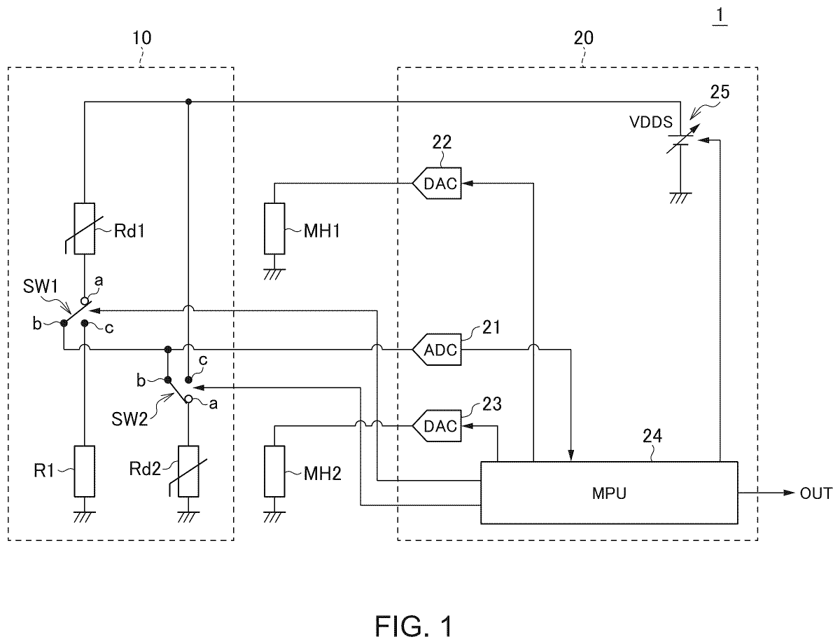

FIG. 1 is a circuit diagram illustrating the configuration of a gas sensor 1 according to a first embodiment of the technology described herein.

As illustrated in FIG. 1, the gas sensor 1 according to the first embodiment includes: a detection circuit 10 including thermistors Rd1, Rd2 and a fixed resistor R1; heaters MH1 and MH2 for heating the thermistors Rd1 and Rd2, respectively; and a control circuit 20 for controlling the detection circuit 10 and heaters MH1 and MH2. Although not particularly limited, the gas sensor 1 according to the present embodiment is a thermal conduction type gas sensor for detecting the concentration of CO₂ gas in the atmosphere.

The detection circuit 10 includes the thermistors Rd1 and Rd2, the fixed resistor R1, and switches SW1 and SW2. The thermistors Rd1 and Rd2 are detection elements made of a material having a negative temperature coefficient of resistance, such as a composite metal oxide, amorphous silicon, polysilicon, or germanium. Both the thermistors Rd1 and Rd2 detect the concentration of CO₂ gas, but have different operating temperatures, as described below. Here, the thermistor Rd1 constitutes a detection element, and the thermistor Rd2 constitutes a reference element.

The switches SW1 and SW2 each have one common node a and two selection nodes b and C, and one of the selection nodes b and c is connected to the common node a. As described later, for both of the switches SW1 and SW2, the selection node b is selected during measurement operation, and the selection node c is selected during dummy heating operation.

As illustrated in FIG. 1, the common node a of the switch SW1 is connected to one end of thermistor Rd1, the selection node b of the switch SW1 is connected to the selection node b of the switch SW2, and the selection node c of the switch SW1 is connected to one end of the fixed resistor R1. The other end of the thermistor Rd1 is connected to a variable power supply 25 that supplies a power supply potential VDDS, and the other end of the fixed resistor R1 is connected to a wiring to which a ground potential GND is supplied. The common node a of the switch SW2 is connected to one end of thermistor Rd2, the selection node b of the switch SW2 is connected to the selection node b of the switch SW1, and the selection node c of the switch SW2 is connected to the variable power supply 25. The other end of the thermistor Rd2 is connected to a wiring to which a ground potential GND is supplied.

As a result, during the measurement operation in which the selection node b is selected, the thermistors Rd1 and Rd2 are connected in series between the variable power supply 25 and the ground. On the other hand, during the dummy heating operation in which the selection node c is selected, the thermistor Rd1 and fixed resistor R1 are connected in series between the variable power supply 25 and the ground, and the thermistor Rd2 is connected in parallel with the thermistor Rd1 and fixed resistor R1. The fixed resistor R1 has a resistance value of 51 kΩ, for example.

The control circuit 20 includes an AD converter (ADC) 21, DA converters (DAC) 22 and 23, an MPU 24, and the variable power supply 25. The AD converter 21 AD-converts a detection signal appearing at the selection nodes b of the switches SW1 and SW2, i.e., appearing at the connection point between the thermistors Rd1 and Rd2 during the measurement operation and supplies the resultant value to the MPU 24. Based on the supplied AD-converted signal, the MPU 24 generates an output signal OUT indicating the concentration of CO₂ gas. Further, the MPU 24 controls the switches SW1 and SW2 to control the connection relation in the detection circuit 10. The DA converters 22 and 23 DA-convert the digital value supplied from the MPU 24 to apply predetermined voltages to the heater resistors MH1 and MH2, respectively. In other words, the heating temperatures of the heater resistors MH1 and MH2 are controlled by the MPU 24. The level of the power supply potential VDDS output from the variable power supply 25 is also controlled by the MPU 24.

The following describes the operation of the gas sensor 1 according to the present embodiment.

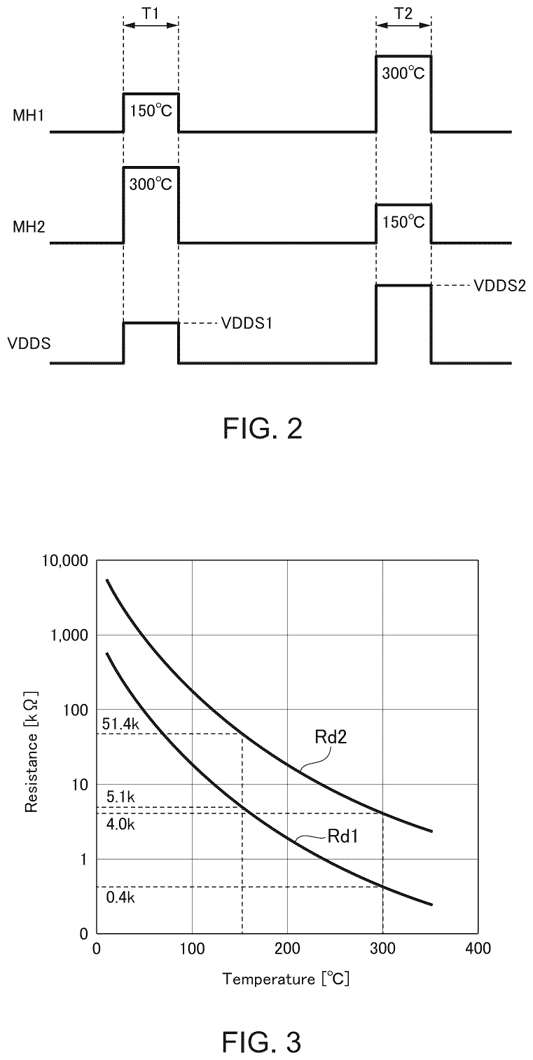

FIG. 2 is a timing chart for explaining the operation of the gas sensor 1 according to the present embodiment.

As illustrated in FIG. 2, the gas sensor 1 according to the present embodiment performs the measurement operation in period T1 and dummy heating operation in period T2. The measurement operation and dummy heating operation are performed alternately.

During the measurement operation performed in period T1, heater resistors MH1 and MH2 are heated to 150° C. and 300° C., respectively, under the control of MPU 24. As illustrated in FIG. 3, the temperature characteristics of thermistors Rd1 and Rd2 are different from each other, and they are designed such that the resistance value of the thermistor Rd1 heated to 150° C. and the resistance value of the thermistor Rd2 heated to 300° C. are close to each other. In the example illustrated in FIG. 3, the resistance value of the thermistor Rd1 heated to 150° C. is 5.1 kΩ, and the resistance value of the thermistor Rd2 heated to 300° C. is 4.0 kΩ. The resistance value of the thermistor Rd1 heated to 150° C. and the resistance value of the thermistor Rd2 heated to 300° C. may be approximately the same.

As described above, since the selection nodes b of the switches SW1 and SW2 are selected during the measurement operation, the thermistors Rd1 and Rd2 are connected in series. Further, the MPU 24 controls the variable power supply 25 to set the power supply potential VDDS during the measurement operation to a first level VDDS1. The first level VDDS1 is 2.7 V, for example.

When CO₂ gas is present in the measurement atmosphere with the thermistor Rd1 as the detection element heated to 150° C., the heat dissipation characteristics of the thermistor Rd1 change according to the concentration. Such a change appears as a change in the resistance value of the thermistor Rd1. On the other hand, even when CO₂ gas is present in the measurement atmosphere with the thermistor Rd2 as the reference element heated to 300° C., the heat dissipation characteristics of the thermistor Rd2 hardly change according to the concentration. Therefore, the change in the resistance value of the thermistor Rd2 heated to 300° C. due to the concentration of CO₂ gas is sufficiently smaller than the change in the resistance value of the thermistor Rd1 heated to 150° C. due to the concentration of CO₂ gas. There is no problem if the resistance value of the thermistor Rd2 heated to 300° C. due to the concentration of CO₂ gas hardly changes.

As a result, the level of the detection signal appearing at the connection point between the thermistors Rd1 and Rd2 changes according to the concentration of CO₂ gas in the measurement atmosphere. The detection signal is supplied to the MPU 24 through the AD converter 21, and the MPU 24 generates the output signal OUT indicating the concentration of CO₂ gas based on the supplied detection signal.

During the dummy heating operation performed in period T2, heater resistors MH1 and MH2 are heated to 300° C. and 150° C., respectively, under the control of the MPU 24. This eliminates the thermal history difference between thermistors Rd1 and Rd2 during the measurement operation performed in period T1.

However, simply reversing the heating temperatures of the thermistors Rd1 and Rd2 during the dummy heating operation with respect to the heating temperatures of the thermistors Rd1 and Rd2 during the measurement operation may cause a slight difference in thermal history due to the self-heating of the thermistors Rd1 and Rd2. That is, the thermistor Rd1 is designed to have a predetermined resistance value when heated to 150° C., and the thermistor Rd2 is designed to have a predetermined resistance value when heated to 300° C., so that when the heating temperatures of the Rd1 and Rd2 are reversed, the resistance value of the thermistor Rd2 becomes significantly higher than the resistance value of the thermistor Rd1. In the example illustrated in FIG. 3, the resistance value of the thermistor Rd1 heated to 300° C. is 0.4 kΩ, and the resistance value of the thermistor Rd2 heated to 150° C. is 51.4 kΩ, which reveals a difference of over a hundred times. Therefore, when the thermistors Rd1 and Rd2 are connected in series even during the dummy heating operation, the self-heating amount of the thermistor Rd2 becomes larger than that of the thermistor Rd1.

Thus, in the present embodiment, the switches SW1 and SW2 are used to switch the connection relation in the detection circuit 10 during the dummy heating operation to thereby make the amounts of currents flowing through the thermistors Rd1 and Rd2 different from each other and set the power supply potential VDDS during the dummy heating operation to a second level VDDS2. The second level VDDS2 is 4.77 V, for example.

When the thermistors Rd1 and Rd2 are made of a material having a negative temperature coefficient of resistance as in the present embodiment, the resistance value of the thermistor Rd2 during the dummy heating operation becomes significantly large, so that, during the dummy heating operation, it is necessary to make the amount of a current flowing through the thermistor Rd1 larger than the amount of a current flowing through the thermistor Rd2. This reduces a difference between the self-heating amount of the thermistor Rd1 during the dummy heating operation and the self-heating amount of the thermistor Rd2 during the dummy heating operation.

For example, while the level of the power supply potential VDDS during the measurement operation is 2.7 V, when the resistance value of the thermistor Rd1 heated to 150° C. is 5.1 kΩ and the resistance value of the thermistor Rd2 heated to 300° C. is 4.0 kΩ, the power consumptions of the thermistors Rd1 and Rd2 during the measurement operation are 0.442 mW and 0.349 mW, respectively. On the other hand, while the level of the power supply potential VDDS during the dummy heating operation is 4.77 V, when the resistance value of the thermistor Rd1 heated to 300° C. is 0.4 kΩ and the resistance value of the thermistor Rd2 heated to 150° C. is 51.4 kΩ, the power consumptions of the thermistors Rd1 and Rd2 during the dummy heating operation are 0.349 mW and 0.442 mW, respectively. In this case, when the heating time of the heater resistors MH1 and MH2 in period T1 and the heating time of the heater resistors MH1 and MH2 in period T2 are made to coincide with each other, not only a difference between the self-heating amount of the thermistor Rd1 during the dummy heating operation and the self-heating amount of the thermistor Rd2 during the dummy heating operation is reduced, but also the self-heating amount of the thermistor Rd1 during the measurement operation and the self-heating amount of the thermistor Rd2 during the dummy heating operation substantially coincide with each other, and the self-heating amount of the thermistor Rd2 during the measurement operation and the self-heating amount of the thermistor Rd1 during the dummy heating operation substantially coincide with each other, thereby eliminating the thermal history difference due to self-heating substantially completely.

However, in the present embodiment, it is not essential that the self-heating amount of the thermistor Rd1 during the measurement operation and the self-heating amount of the thermistor Rd2 during the dummy heating operation substantially coincide with each other and that the self-heating amount of the thermistor Rd2 during the measurement operation and self-heating amount of the thermistor Rd1 during the dummy heating operation substantially coincide with each other, but it is sufficient that the difference in self-heating amount is reduced by independently controlling the amounts of currents flowing through the thermistors Rd1 and Rd2 during the dummy heating operation. Therefore, it is also not essential to switch the level of the power supply potential VDDS between the measurement operation and the dummy heating operation.

Further, it is not essential to make the amount of a current flowing through the thermistor Rd1 larger than the amount of a current flowing through the thermistor Rd2 during the dummy heating operation, but in a case where conditions are different from those set in the present embodiment, such as when the thermistors Rd1 and Rd2 are made of a material having a positive temperature coefficient of resistance, the amount of a current flowing through the thermistor Rd1 may be made smaller than the amount of a current flowing through the thermistor Rd2 during the dummy heating operation.

Further, it is not essential that the heating temperatures of the thermistors Rd1 and Rd2 during the dummy heating operation are strictly reversed with respect to the heating temperatures of the thermistors Rd1 and Rd2 during the measurement operation, but the heating temperatures of the thermistors Rd1 and Rd2 during the dummy heating operation may be set considering heating time, environmental temperature, self-heating amount, and the like as long as the thermal history difference between the thermistors Rd1 and Rd2 is reduced.

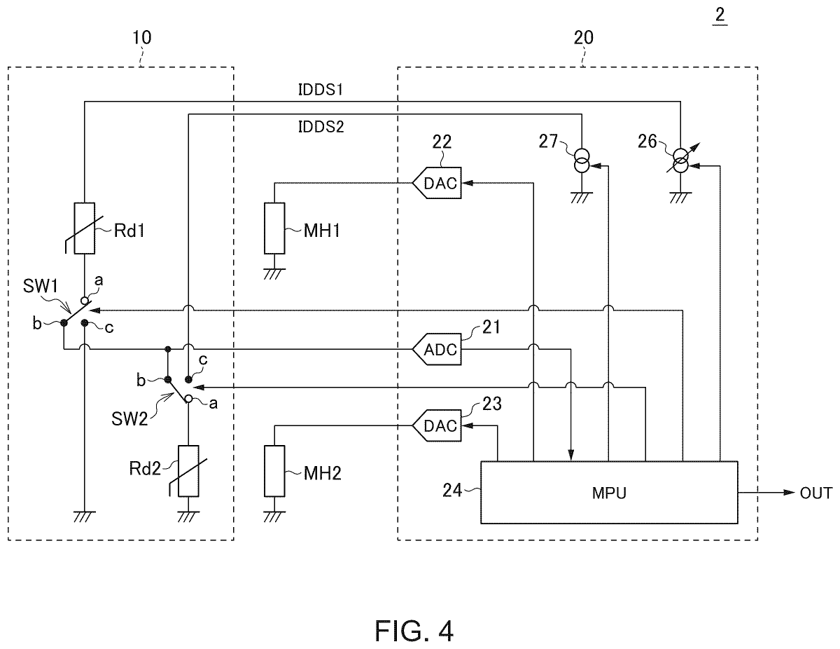

FIG. 4 is a circuit diagram illustrating the configuration of a gas sensor 2 according to a second embodiment of the technology described herein.

As illustrated in FIG. 4, the gas sensor 2 according to the second embodiment differs from the gas sensor 1 according to the first embodiment in that the fixed resistor R1 and variable power supply 25 are omitted and, instead, a variable current source 26 and a fixed current source 27 are included in the control circuit 20. Other basic configurations are the same as those of the gas sensor 1 according to the first embodiment, so the same reference numerals are given to the same elements, and overlapping description will be omitted.

The variable current source 26 is connected to the other end of the thermistor Rd1, and the fixed current source 27 is connected to the selection node c of the switch SW2. As a result, during the measurement operation in which the selection node b is selected, the thermistors Rd1 and Rd2 are connected in series to the variable current source 26. On the other hand, during the dummy heating operation in which the selection node c is selected, the thermistor Rd1 is connected to the variable current source 26, and the thermistor Rd2 is connected to the fixed current source 27.

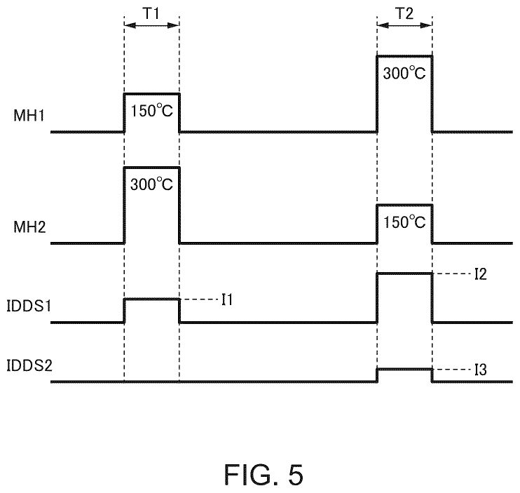

FIG. 5 is a timing chart for explaining the operation of the gas sensor 2 according to the present embodiment.

As illustrated in FIG. 5, in the present embodiment as well, the heater resistors MH1 and MH2 are heated to 150° C. and 300° C., respectively, during the measurement operation performed in period T1, and the heater resistors MH1 and MH2 are heated to 300° C. and 150° C., respectively, during the dummy heating operation performed in period T2. Further, during the measurement operation, the amount of an operating current IDDS1 supplied from the variable current source 26 is set to I1. Accordingly, during the measurement operation, the amount of a current flowing through each of the thermistors Rd1 and Rd2 is I1. The current amount I1 is 0.293 mA, for example. When the resistance value of the thermistor Rd1 heated to 150° C. is 5.1 kΩ, and the resistance value of the thermistor Rd2 heated to 300° C. is 4.0 kΩ, the power consumptions of the thermistors Rd1 and Rd2 during the measurement operation are 0.442 mW and 0.349 mW, respectively.

On the other hand, during the dummy heating operation, the amount of operating current IDDS1 supplied from the variable current source 26 is set to I2. The amount of the operating current IDDS2 supplied from the fixed current source 27 is I(<I2). As a result, during the dummy heating operation, the amount of a current flowing through the thermistor Rd1 is 12, and the amount of a current flowing through the thermistor Rd2 is 13. The current amount I2 is 0.927 mA, for example, and the current amount I3 is 0.093 mA, for example. When the resistance value of the thermistor Rd1 heated to 300° C. is 0.4 kΩ, and the resistance value of the thermistor Rd2 heated to 150° C. is 51.4 kΩ, the power consumptions of the thermistors Rd1 and Rd2 during the dummy heating operation are 0.349 mW and 0.442 mW, respectively.

Therefore, when the heating time of the heater resistors MH1 and MH2 in period T1 and the heating time of the heater resistors MH1 and MH2 in period T2 are made to coincide with each other, the self-heating amount of the thermistor Rd1 during the measurement operation and the self-heating amount of the thermistor Rd2 during the dummy heating operation substantially coincide with each other, and the self-heating amount of the thermistor Rd2 during the measurement operation and the self-heating amount of the thermistor Rd1 during the dummy heating operation substantially coincide with each other.

As exemplified in the second embodiment, different currents may be made to flow through the thermistors Rd1 and Rd2 using a plurality of current sources.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although the measurement target gas is CO₂ gas in the above embodiments, the present invention is not limited to this. Further, the sensor part used in the present invention need not necessarily be a thermal conduction type sensor, but may be a sensor of other types such as a catalytic combustion type. As an example, when the measurement target gas is CO gas, a catalytic combustion type sensor part can be used.

The technology according to the present disclosure includes the following configuration examples, but not limited thereto.

A gas sensor according to the present disclosure includes: a detection circuit including a first thermistor and a second thermistor; a first heater configured to heat the first thermistor; a second heater configured to heat the second thermistor; and a control circuit configured to control a connection relation in the detection circuit and heating temperatures of the first and second heaters. The control circuit is configured to, in a first period, generate an output signal indicating a concentration of a gas to be measured based on a detection signal appearing at a connection point between the first thermistor and the second thermistor by connecting the first and second thermistors in series and heating the first and second heaters such that the heating temperature of the second heater is higher than the heating temperature of the first heater. The control circuit is configured to, in a second period, heat the first and second heaters such that the heating temperature of the first heater is higher than the heating temperature of the second heater and switch the connection relation so as to make an amount of a current flowing through the first thermistor and an amount of a current flowing through the second thermistor different from each other. This can reduce a difference in self-heating amount between the first and second thermistors in the second period.

In the above gas sensor, in the first period, the control circuit may be configured to heat the first heater to a first temperature and heat the second heater to a second temperature, and in the second period, the control circuit may be configured to heat the first heater to the second temperature and heat the second heater to the first temperature. This can reduce a difference in thermal history received by the heaters.

In the above gas sensor, a heating time of the first and second heaters in the first period and a heating time of the first and second heaters in the second period may be equal to each other. This can further reduce a difference in thermal history received by the heaters.

In the above gas sensor, each of the first and second thermistors may contain a material having a negative temperature coefficient of resistance, and the control circuit may be configured to switch the connection relation in the second period so as to make the amount of a current flowing through the first thermistor larger than the amount of a current flowing through the second thermistor. This can reduce the difference in self-heating amount even when the second thermistor is higher in resistance than the first thermistor in the second period.

In the above gas sensor, the detection circuit may further include a fixed resistor and, in the second period, the control circuit may be configured to connect the first thermistor and the fixed resistor in series and connect the second thermistor in parallel with the first thermistor and fixed resistor. This can adjust the amount of a current flowing through the first thermistor in the second period.

In the above gas sensor, the control circuit may configured to apply a first voltage to a series circuit including the first and second thermistors in the first period and apply a second voltage different from the first voltage to a series circuit including the first thermistor and fixed resistor in the second period. This can further reduce the difference in self-heating amount between the first and second thermistors in the second period.

In the above gas sensor, the control circuit may include a first current source and a second current source, in the first period, the control circuit may be configured to activate the first current source to supply a current to a series circuit including the first and second thermistors, and, in the second period, the control circuit may be configured to activate the first current source to supply a current to the first thermistor and activate the second current source to supply a current to the second thermistor. This can easily supply different currents to the first and second thermistors in the second period.

In the above gas sensor, the control circuit may be configured to control a current flowing through the first thermistor and a current flowing through the second thermistor such that a self-heating amount of the first thermistor in the first period and a self-heating amount of the second thermistor in the second period coincide with each other and that a self-heating amount of the second thermistor in the first period and a self-heating amount of the first thermistor in the second period coincide with each other. This can eliminate substantially completely the thermal history difference due to self-heating of the first and second thermistors.

What is claimed is:

1. A gas sensor comprising:
a detection circuit including a first thermistor and a second thermistor;
a first heater configured to heat the first thermistor;
a second heater configured to heat the second thermistor; and
a control circuit configured to control a connection relation in the detection circuit and heating temperatures of the first and second heaters,
wherein the control circuit is configured to:
in a first period, generate an output signal indicating a concentration of a gas to be measured based on a detection signal appearing at a connection point between the first thermistor and the second thermistor by connecting the first and second thermistors in series and heating the first and second heaters such that the heating temperature of the second heater is higher than the heating temperature of the first heater; and
in a second period, heat the first and second heaters such that the heating temperature of the first heater is higher than the heating temperature of the second heater and switch the connection relation so as to make an amount of a current flowing through the first thermistor and an amount of a current flowing through the second thermistor different from each other.

2. The gas sensor as claimed in claim 1,
wherein, in the first period, the control circuit is configured to heat the first heater to a first temperature and heat the second heater to a second temperature, and
wherein, in the second period, the control circuit is configured to heat the first heater to the second temperature and heat the second heater to the first temperature.

3. The gas sensor as claimed in claim 2, wherein a heating time of the first and second heaters in the first period and a heating time of the first and second heaters in the second period are equal to each other.

4. The gas sensor as claimed in claim 1,
wherein each of the first and second thermistors contains a material having a negative temperature coefficient of resistance, and
wherein the control circuit is configured to switch the connection relation in the second period so as to make the amount of a current flowing through the first thermistor larger than the amount of a current flowing through the second thermistor.

5. The gas sensor as claimed in claim 4,
wherein the detection circuit further includes a fixed resistor, and
wherein, in the second period, the control circuit is configured to connect the first thermistor and the fixed resistor in series and connect the second thermistor in parallel with the first thermistor and fixed resistor.

6. The gas sensor as claimed in claim 5,
wherein the control circuit is configured to apply a first voltage to a series circuit including the first and second thermistors in the first period, and
wherein the control circuit is configured to apply a second voltage different from the first voltage to a series circuit including the first thermistor and fixed resistor in the second period.

7. The gas sensor as claimed in claim 4,
wherein the control circuit includes a first current source and a second current source,
wherein, in the first period, the control circuit is configured to activate the first current source to supply a current to a series circuit including the first and second thermistors, and
wherein, in the second period, the control circuit is configured to activate the first current source to supply a current to the first thermistor and activate the second current source to supply a current to the second thermistor.

8. The gas sensor as claimed in claim 1, wherein the control circuit is configured to control a current flowing through the first thermistor and a current flowing through the second thermistor such that a self-heating amount of the first thermistor in the first period and a self-heating amount of the second thermistor in the second period coincide with each other and that a self-heating amount of the second thermistor in the first period and a self-heating amount of the first thermistor in the second period coincide with each other.
